(19) United States
(12) Patent Application Publication
(10) Pub. No.: US 2009/0306019 A1
(43) Pub. Date: Dec. 10, 2009

(54) Compositions for treatment of eosinophilia and related disorders

(75) Inventor: Avner Shenfeld, Rehovot (IL)

Correspondence Address: THE NATH LAW GROUP, 112 South West Street, Alexandria, VA, 22314, US

(73) Assignee: MODUS BIOLOGICAL MEMBRANES LTD., Rehovot (IL)

(21) Appl. No.: 11/991,173

(22) PCT Filed: Aug. 30, 2006
(86) PCT No.: PCT/IL2006/001004

§ 371 Date: Jul. 10, 2009

Related U.S. Application Data
(60) Provisional application No. 60/712,449, filed on Aug. 31, 2005.

(51) Int. Cl. A61K 31/661; A61P 7/00

(52) U.S. Cl. 514/120

(57) ABSTRACT

The present invention discloses compositions comprising phosphatidic acid and uses thereof for the treatment of subjects suffering from eosinophilia or related disorders such as asthma and/or the prevention of these disorders in subjects having environmental or genetic predisposition thereto.

### FIELD OF THE INVENTION

This invention relates generally to the treatment of eosinophilia and related disorders such as asthma.

### BACKGROUND OF THE INVENTION

The eosinophil is a terminally differentiated, end-stage leukocyte that resides predominantly in submucosal tissue and is recruited to sites of specific immune reactions, including allergic diseases. The eosinophils mainly settle in the tissue where their number is about one hundred times higher than in the blood.

The eosinophils contain large specific granules which are the principal identifying feature of eosinophils, each containing four distinct cationic proteins which exert a range of biological effects on host cells and microbial targets: major basic protein (MBP), oesinophil cationic protein (ECP), eosinophil derived neurotoxin (EDN), and eosinophil peroxidase (EPO).

These proteins have major effects in contributing to tissue dysfunction and damage in eosinophil related inflammatory and allergic diseases. As MBP lack enzymatic activity, one mechanism whereby this highly cationic polypeptide may exert its toxic activities is by interactions with lipid membranes leading to their derangement. Both MBP and EPO have been shown to act as selective allosteric inhibitors of agonist binding to M2 muscarinic receptors. Thus, these proteins may contribute to M2 receptor dysfunction and enhance vagally mediated bronchoconstriction in asthma.

Only recently has it been recognized that eosinophils are capable of elaborating cytokines which include those with potential autocrine growth-factor activities for eosinophils and those with potential roles in acute and chronic inflammatory responses. Three cytokines have growth-factor activities for eosinophils: granulocyte-macrophage colony-stimulating factor (GM-CSF), IL-3 and IL-5. Other cytokines produced by human eosinophils that may have activities in acute and chronic inflammatory responses include IL-1α, IL-6, IL-8, TNF-α and both transforming growth factors, TGF-α and TGF-β.

Eosinophils also participate in hypersensitivity reactions, especially through two lipid inflammatory mediators, leukotriene C⁴ (LTC⁴) and platelet activating factor (PAF). Both mediators contract airway smooth muscle, promote the secretion of mucus, alter vascular permeability and elicit eosinophil and neutrophil infiltration. Thus, once stimulated, eosinophils can serve as a local source of specific lipid mediators as well as induce the release of mediators from mast cells and basophils.

The processes that lead to the accumulation of eosinophils within tissue sites of specific inflammation, as for other leukocytes, involve numerous sequential interactions that enable eosinophils to adhere to and then transmigrate through the endothelium and to respond to local chemoattractants. The adhesion of eosinophils to endothelium include CD18-dependent pathways, interaction between E-selectin and P-selectin and adherence to VCAM by means of very late antigen 4 (VLA-4) expressed on the eosinophil.

The prevalence of allergic diseases in general and asthma in particular has been rising continuously in Western countries. Approximately, fifteen million individuals in the USA have asthma and the disease is the cause of more than five thousand deaths annually in the USA alone. In children asthma represents the most prevalent chronic disease causing major health expenditure which stems mainly from the necessity for emergency treatments and hospitalization. Additionally, asthma is the most common cause of school and work absenteeism in the USA.

The current immunologic paradigm, trying to explain the underlying mechanism of asthma is that a shift occurs in the T-helper cell type 1/type 2 (Th-1/Th-2) balance towards the later, while an effective immune response requires a Th-1 dominance. Various explanations for this shift, such as the “hygiene hypothesis” have been also proposed.

A Th-1 type profile is characterized by the secretion mainly of interferon gamma (IFNγ) and interleukin 2 (IL-2), while Th-2 type cells secrete cytokines, such as IL-4, which promotes an IgG to IgE switch, IL-5 which induces eosinophilia and IL-13 which overlaps some IL-4 functions and promotes subepithelial fibrosis and mucus plug formation. IL-10 regulates this balance, thus inhibiting Th-1 responses in favor of the Th-2 type. Recently, a group of additional Th-subtypes has been identified, designated as Treg cells, which secrete predominantly the inflammation suppressive cytokines IL-10 and transforming growth factor beta (TGFβ). This observation could call for a revision of the possibly too simplistic Th-1/Th-2 paradigm as the sole explanation of the immunologic mechanism of asthma.

Thus, a possible “reprogramming” of the immune system by redirecting the immune response towards rebalancing away from the chronic, Th-2 type driven inflammation, may be beneficial in these conditions. Indeed recently it has been shown that a normalization of the secretion of IFNγ is beneficial in human asthma and a polymorphism in the IFNG gene may be associated with an increased asthma incidence in an Indian population.

Some phospholipids (PLs) have been shown to reconstitute brain cells membrane fluidity, which is altered in chemical substance abuse addiction and in the aging process, thereby interfering with inter-cell signalization. However, little is known about the specific effect of PLs on the function of immunocytes, although some lyso-phospholipids (Ly-PL) have been shown to induce activation and proliferation of some cell lineages, such as a Jurkat T-cell line.

Current treatment options for eosinophilia related disorders such as asthma include medications that control the airway inflammatory component of the disease and rapid relief medications that counteract bronchospasm. The most commonly prescribed medications are beta 2-adrenoreceptor agonists such as epinephrine, isoproterenol, albuterol, and salmeterol; xanthines such as caffeine and theophylline; anti-inflammatory agents such as disodium cromoglycate (DSCG), nedocromil sodium; antihistamines such as ketotifen; and corticosteroids such as prednisolone.

There are several disadvantages to using these medications. There is a potential lack of effective sustained action; there are side effects associated with prolonged use of these medications, specifically in the case of corticosteriods and beta-adrenergic agents; there is a progressive loss of sensitivity to the treatments after prolonged use; there is limited efficacy of any of these treatments in severe cases of asthma; the treatments are not necessarily selective, thus affecting other organs aside from the lungs, and as such are at times considered a potential risk.

There is therefore a long felt need for a medicament for treating eosinophilia and related conditions such as asthma which is less toxic, more specific and lacking the deficiencies of the known anti-asthma medicaments.

### SUMMARY OF THE INVENTION

The present invention is based on the surprising finding that phosphatidic acid (PA) is efficient in reducing eosinophil concentration in tissues and thus in affecting treatment of eosinophilia or related states such as asthma. The invention is additionally based on the fining that the PA composition is effective in reducing the occurrence of the asthmatic attacks.

Thus, the present invention generally provides methods for the treatment and/or the prevention of asthma and other disorders associated with increased concentrations of eosinophils and further provides compositions useful in the treatment and prevention of such diseases and disorders.

In a first aspect of the invention, there is provided a pharmaceutical composition for the treatment or prevention of a disease or disorder in a tissue associated with an increase of eosinophils in said tissue, said composition comprising an effective amount of phosphatidic acid (PA) and a pharmaceutically acceptable carrier, diluent or excipient.

In one embodiment, said disease or disorder is eosinophilia or a related state.

In another embodiment, said disease or disorder is of the respiratory tract, preferably asthma or an asthma related state, such as Eosinophilic Pneumonitis.

The term “tissue”, as used herein refers to a whole or part of a tissue being part of a subject's body (e.g., mucosa, the lining of the respiratory tract), the surrounding of said tissue, part of an organ or a whole organ (such as lungs, kidneys, gut), including blood, and a part of said tissue being outside of the subject's body (in vitro or ex vivo). Preferably, the tissue is a tissue of the respiratory tract such as the mucosa and lungs.

The expression “a disease or disorder of a tissue” refers to an abnormal condition of said organ, as defined, which is associated with a chronic or acute increase in the concentration of eosinophils.

The term “asthma” relates generally to a condition of the respiratory tract characterized by widespread, reversible narrowing of the airways (bronchoconstriction) and increased sensitivity (hypersensitivity) of the airways to a variety of stimuli. Within the scope of the present invention, the term refers to such a disease which is triggered by various stimuli such as: inhaled allergens, inhaled low molecular weight agents to which a subject has become sensitized, viral or mycoplasma respiratory infections and oxidizing gases such as ozone and nitrogen oxides, or any other triggers which are associated with the induction of airway inflammation.

By the expression “asthma-related state”, as used herein, is meant the pro-asthmatic phenotype which is observed in airway smooth muscle cells, characterized by increased contraction and decreased relaxation of the airway tissue when it is exposed to high IgE-containing atopic asthmatic serum or exogenous IgE, compared with airway tissue which has not been exposed to IgE. This is usually associated with chronic bronchial inflammation, which may lead to bronchial hypersensitivity.

The term “eosinophilia” relates generally to conditions in which abnormally high amounts of eosinophils are found in either the blood or in body tissues. Eosinophilia occurs in a wide range of conditions. Its commonest cause worldwide is parasitic infection but it may also occur in allergic diseases such as asthma and hay fever, and in relation to related states such as Eosinophilic gastroenteritis and esophagitis, or common skin diseases and reactions to medicines. Other causes include: lung diseases, such as Loeffler's syndrome, vasculitis (inflammation of blood vessels), such as Churg-Strauss syndrome, some tumors, such as lymphoma, liver cirrhosis, some antibody deficiencies; skin diseases, such as dermatitis herpetiformis, and hypereosinophilic syndrome.

Normally, eosinophils constitute 1 to 3% of the peripheral blood leukocytes, at a count of 350 to 650 per cubic millimeter. Eosinophilia can be categorized as mild (less than 1500 eosinophils per cubic millimeter), moderate (1500 to 5000 per cubic millimeter), or severe (more than 5000 per cubic millimeter). As the present invention demonstrates, the use of PA may reduce the accumulation of eosinophils and thus reduce the severity of the eosinophilia.

Eosinophilia may be primary or secondary. In primary eosinophilia, the increased production of eosinophils is due to an abnormality in a hematopoietic stem cell as, for example, in eosinophilic leukemia. In secondary eosinophilia, the increased production of eosinophils is a reactive process driven by cytokines, as is the case in allergy.

The term “treatment of a disease” as used herein refers to the administering of a therapeutic amount of the PA composition of the present invention which is effective to ameliorate undesired symptoms associated with the disease, to prevent the manifestation of such symptoms before they occur, e.g., by reducing or preventing the accumulation of eosinophils in the tissue, to slow down the progression of asthma attacks for example by brining about such a reduction in eosinophil concentration, to slow down the deterioration of symptoms, to enhance the onset of remission period or to prolong asthma-free periods, slow down the irreversible damage caused in the progressive chronic stage of the disease, to delay the onset of said progressive stage, to lessen the severity or cure the disease or any state associated therewith, to bring about rapid recovery, or to prevent the disease form occurring or any combination of the above.

The term “prevention” refers to (a) the reduction of the instances of the diseases and/or disorders mentioned herein; (b) the reduction of the prevalence of the disease and/or disorder in a treated population and the reduction in the duration of said disease and/or disorder, and (c) the reduction of the incidence of chronic incapacity or recurrences in the treated population.

The composition of the present invention typically comprises at least about 5% PA.

In one embodiment, the composition comprises between about 5% to about 75%. In another embodiment of the present invention, the PA composition comprises between about 7% to about 50% PA or 10% to about 50% PA.

Preferably, the composition comprises between about 15% to about 30% PA. More preferably, the composition comprises between about 15% to about 25% PA. Most preferably, the compositions comprise about 16%, 17%, 18%, 19%, 20%, 21% or 22% PA.

In addition to the PA, the composition of the invention may also comprise other phospholipids, such as phophatidylcholine, phosphatidylnositol, phosphatidylethanol amine, phosphatidylglycerol and/or triglycerides.

The composition may also contain pharmaceutically acceptable carriers and other components such as adjuvants, e.g. aqueous suspensions of aluminum and magnesium hydroxides, antioxidants such as beta-carotene and vitamin E or C, other phospholipids from an animal or vegetable source, such as phosphatidylserine, and other agents which are known in the treatment of asthma and may be included in the composition in order to achieve a better ant-asthmatic effect.

In yet another aspect of the present invention there is provided a use of PA or a PA containing mixture for the preparation of a pharmaceutical composition for the treatment or prevention of a disease or disorder in a tissue associated with an increase of eosinophils in said tissue (i.e. eosinophilia or a related state). Preferably, the disease or disorder is associated with the respiratory tract. Most preferably, the disease or disorder is asthma or asthma related state.

In one embodiment, said composition is for the treatment of asthma, asthma related state or for preventing induction of asthmatic state in a mammalian subject. Preferably, said asthma or asthma-related state is chronic bronchial inflammation.

In yet another aspect of the present invention, there is provided a method for the treatment or prevention of a disease or disorder in a tissue associated with an increase of eosinophils in said tissue, said method comprising administering to a subject in need thereof an effective amount of a composition of the present invention.

In one embodiment, said subject, preferably a human subject, suffers from eosinophilia or a related state. In another embodiment, said subject suffers from asthma or a related state.

The present invention also provides a method for preventing an increase in eosinophils in a tissue of a subject, said method comprising administering to said subject an effective amount of a composition of the present invention.

In one embodiment, said subject, preferably a human subject, has a genetic or environmental predisposition to develop eosinophilia or a related state. In another embodiment, said subject has a genetic or environmental predisposition to develop asthma or a related state.

Further, the present invention provides a method for preventing or minimizing the accumulation of eosinophils in a tissue, preferably of the respiratory tract, of a subject suffering from eosinophilia or a related state, or having an environmental or genetic predisposition to develop eosinophilia or a related state, said method comprising administering to said subject an effective amount of a composition of the invention.

Preferably, the tissue is a tissue of the respiratory tract such as the mucosa and the lungs.

The composition of the invention is typically administered in a unit dose form containing an effective amount of PA. The expression “effective amount” for purposes herein is determined by such considerations as may be known in the art. The amount must be effective to achieve the desired therapeutic effect as described above, e.g., reduce the concentration of eosinophils in a tissue, depending, inter alia, on the type and severity of the condition to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the distribution profile of the PA within the body, a variety of pharmacological parameters such as half life in the body, on undesired side effects, if any, on factors such as age and gender, etc.

The pharmaceutically acceptable carriers, for example, vehicles, adjuvants, excipients, or diluents, are well-known to those who are skilled in the art and are readily available to the public. It is preferred that the pharmaceutically acceptable carrier be one which is chemically inert to the active compound (PA) and one which has no detrimental side effects or toxicity under the conditions of use. The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intramuscular, interperitoneal, rectal, and vaginal administration are merely exemplary and are in no way limiting.

Formulations suitable for oral administration can consist of (a) liquid solutions, such as an effective amount of PA dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the PA in a flavor, usually sucrose and acacia, as well as pastilles comprising the active ingredient in an inert base, such as gelatin and glycerin, or sucrose and acacia, emulsions, gels, and the like containing, in addition to the active ingredient, such carriers as are known in the art.

The compositions of the present invention, containing PA alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The composition of the invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, canola oil, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The requirements for effective pharmaceutical carriers for injectable compositions are well known to those of ordinary skill in the art, see Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Additionally, the compositions of the present invention may be made into suppositories by mixing with a variety of bases, such as emulsifying bases or water-soluble bases.

Administration via the oral cavity or by inhalation is preferred.

Other possible formulations such as nanoparticles and liposomes may be also used for the delivery of the composition of the invention.

### DETAILED DESCRIPTION OF EXEMPLARY EMBODIMENTS

The phosphatidic acid (PA) may be produced by a number of different methods, e.g. by chemical synthesis or by natural extraction. In accordance with one embodiment, the PA is a product of partial hydrolysis of lecithin phospholipids, e.g. using phospholipase-D from peanuts. It is a complex mixture of hexane-soluble substances which consists of phosphatidic acid, other phospholipids such as phophatidylcholine, phosphatidylnositol, phosphatidylethanolamine, phosphatidylglycerol and triglycerides. In the enriched mixture, herein referred to as the “primary PA mixture” the total PA is between 40% and 50%. The concentration given in % units means to denote the number of weight units of the ingredient per 100 weight units of the entire composition (w/w).

The primary PA mixture is preferably obtained from natural phospholipids preparation by enzymatic treatment using synthetic or natural source comprising the enzyme phospholipase-D. One such preparation is disclosed in U.S. Pat. No. 6,051,564 to the same inventors of the present invention.

Typical examples of natural phospholipids preparations useful for the preparation of the PA mixture are, without being limited thereto, soya lecithin, and egg yolk. Typically, the phospholipase-D source is, without being limited thereto, peanuts, cabbage, bacteria or fractions derived therefrom.

The compositions of the present invention are typically prepared by admixing the primary PA mixture with at least one suitable solubilizing agent that is capable of dissolving the highly viscous or at times pasty primary PA mixture. The composition may also comprise other ingredients such as, and without being limited thereto, vitamins, antioxidants, preservatives, stabilizers and the like. The overall concentration of the PA in the compositions of the present application is typically between at least about 7% and at least about 75%. The term “at least about” as used with reference to the concentration of PA in the compositions of the present invention, for example in the expression “at least about 7%”, is meant to mean 7% PA+/−2%, namely compositions comprising 5% also fall within the scope of the present invention.

In the examples disclosed herein, capsules made of gelatin (Type LB, Gel Formula 3A), size 22, oblong, brown colored (151A) were used. Each capsule contained: PA mixture, oil and vitamin E to a total weight of 1000 mg. Several different formulations were used:

Formulation 1 contained 7% PA: 400 mg canola oil, about 50-80 mg phosphatidic acid, Vitamin E and about 440 mg of other phospholipids.

Formulation 2 contained 16% PA: 400 mg canola oil, about 160 mg phosphatidic acid, 3.5 mg Vitamin E, and about 436 mg other phospholipids.

Formulation 3 contained 18% PA: 400-500 mg canola oil or peanut oil, about 180 mg phosphatidic acid, 3.5 mg Vitamin E, and about 300-407 mg other phospholipids.

Formulation 4 contained 22% PA: 400-500 mg canola oil, about 200-220 mg phosphatidic acid, 3.5 mg Vitamin E, and about 300-400 mg other phospholipids.

### Example 1

### Double-Blind Clinical Trial

Blood samples were drawn from a group of smoking volunteers who had been administered with a composition comprising 18% PA for a period of 3 weeks, twice daily. Results from the tested blood samples indicated that secretion of the cytokine interferon gamma (IFNγ), from peripheral blood mononuclear cells (PBMC) isolated from venous heparinized blood on isopaque gradients was increased in the treated group, as compared to the control group, as shown in Table 1. As IFNγ is considered to be the hallmark of a Th-1 type response, its increase shows that the PA was able to shift the Th-1/Th-2 balance towards the former and thus reduce the allergic state. This finding may indicate the potential of the PA composition to prevent the development of asthma in predisposed individuals.

TABLE 1
IFNγ Secretion by PBMC.

| | PA composition | Control | P |
|---|---|---|---|
| Pre | 0.87 +/− 0.39 | 1.31 +/− 1.44 | NS |
| Post | 1.48 +/− 1.75 | 0.77 +/− 0.49 | <0.05 |

Note:
The values represent means of arbitrary units +/−SD of the treated vs. the control groups.

### Example 2

### Effect of the Pharmaceutical Composition Containing PA as Compared to Lecithin on Cytokine Secretion

In a series of in-vitro experiments, the effects of a composition comprising PA or lecithin, in a sonicated emulsified formulation, were studies on the proliferation and the secretion of cytokines such as IFNγ and IL-10, by cultured PBMC of allergic and asthmatic patients, as compared to normal controls. Cells were cultured for 24 hrs in 200 μl wells of microtiter plates, filled with medium with or without a stimulant, such as PPD or mitogen. Cytokines were measured in supernatants of the cultures by standard ELISA assays (R&D). A trend was shown towards an enhanced proliferation and an increased secretion of IFNγ by PPD-stimulated (protein purified derivative of mycobacteria) cells in the presence of the PA composition as shown in Table 2

These results suggest that the PA composition may be a moderate enhancer of IFNγ secretion, which is the feature needed therapeutically, because large quantities of IFNγ may be pro-inflammatory, therefore deleterious. A recent finding in a mouse asthma model corroborates this assumption by showing that the administration of very low doses of IFNγ block the homing of inflammatory Th-2 cells into the lungs and downregulate bronchial inflammation in a mouse model of asthma (Flaishon L, Topilski I, Shoseyov D et al, Cutting edge: Anti-inflammatory properties of low levels of IFN gamma J Immunol, 2002, 168: 3707-11).

This is in agreement with the modest increases in IFNγ secretion by cells cultured with the PA composition of the invention.

TABLE 2
In-vitro Effect of a PA composition Sonicates on Cytokine Secretion.

| | |
|---|---|
| | PA |
| Medium | composition |

| | | IL-10 | INFγ | IL-10 | INFγ |
|---|---|---|---|---|---|
| Control | Medium | <5 | 30 | 25 | 30 |
| | PPD | 8 | 568 | 70 | 653 |
| | PHA | 159 | >1000 | 217 | 1000 |
| Asthma | Medium | <5 | 30 | 70 | 84 |
| | PPD | 11 | >1000 | 138 | >1000 |
| | PHA | 149 | <5 | 212 | 30 |
| | LPS | 425 | 996 | 764 | 703 |
| Urticaria | Medium | <5 | <30 | 21 | 67 |
| | PPD | 15 | 33 | 43 | 205 |
| | PHA | 217 | 983 | 170 | 998 |
| | LPS | <5 | 942 | 15 | >1000 |

Note:
The lowest detection cut-off level of the IL-10 and IFNγ kits was <5 and <30 pg/ml, respectively, while the highest was >1000 pg/ml for both kits.
PPD—protein purified derivative of mycobacteria;
PHA—phytohemagglutinin;
LPS—lypopolysaccharide.

### Example 3

### Acute Allergic Inflammation Model in Animals

Nineteen Brown Norwegian (BN) rats were sensitized to short ragweed pollen by a single subcutaneous injection which was followed by 2 weeks of oral gavage administration of the PA composition or canola oil and then a single aerosolized allergen challenge. Such a challenge resulted in an eosinophilic inflammatory infiltrate that peaked within 2-3 days. Evaluations of the effectiveness of the compositions of the invention were done 48 hours after challenge, using BAL cell counts (total and differential) as the primary outcome variable.

The PA composition of the invention was administered by oral gavage in a volume of 0.5 ml, once daily in the morning, using a device designed for that purpose. The PA composition was administered in doses of 100 mg/kg (20-25 mg for rats in the 200-250 g weight range). Treatments were 7 days prior to challenge and were continued passed the day of challenge to a day thereafter.

The rats tolerated the gavages well, with no abnormal stools noted during the treatments, and with no fluctuations in weight. After the 7^{th} dose, 6 rats from the canola oil group were exposed to PBS aerosol for 20 minutes (C-PBS group), 6 rats from the canola oil group were exposed to 2% aerosolized ragweed pollen extract for 20 minutes (C-RW group), and the 7 rats which were treated with the PA composition of the invention, were also exposed to ragweed extract aerosol (L-RW group). The rats received an additional gavage treatment the day after challenge.

Two days after aerosol challenge, the rats were anesthetized with urethane, 1.5 g/kg i.p., and euthanized by cutting the abdominal aorta after a blood sample was obtained. After placing a tracheal cannula and clamping the left mainstem bronchus, the right lung was lavaged 5 times with PBS.

BAL cells total leukocytes were counted, and a cytospin slide was prepared to determine the relative % of lymphocytes, macrophages, eosinophils and neutrophils. Serum was frozen, and the left lungs were fixed in formalin, and preserved in Prefer fixative for possible additional studies.

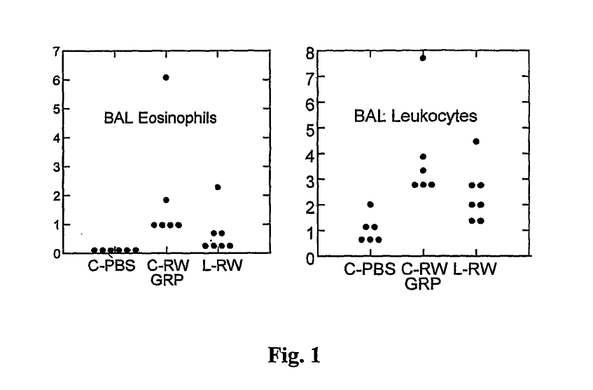

The ragweed-challenged rats had increased BAL leukocytes (lymphocytes, eosinophils and neutrophils significantly increased relative to PBS-challenged rats). However, in the PA-treated group, the total number of BAL leukocytes (p=0.046, Mann-Whitney), and the number of BAL eosinophils (p=0.022, Mann-Whitney) were significantly lower as compared with ragweed-challenged rats from the canola oil group, as shown in FIG. 1 (numbers of total leukocytes and eosinophils (×10⁶ cells) recovered from right lung bronchoalveolar lavage in individual rats of each treatment group). Numbers of BAL lymphocytes, macrophages and neutrophils were not altered by the PA treatment.

### Example 4

### Investigation of the Immunomodulatory Potential of a PA Composition

In-vitro effects of the PA compositions on some immune parameters of PBMC are measured on about 30 asthmatic/allergic patients, preferentially reactive to house-dust mites and who are not treated systemically by steroids, as well as on 15 normal controls from hospital personnel.

Cultured cells are stimulated with PPD, a purified mite antigen, and LPS, in the presence or absence of various concentrations of a PA composition and control phospholipids during 24-48 hrs. Assays include measurement of cytokine secretion before and after treatment. The secreted cytokine proteins are measured in the cell supernatants with conventional sensitive ELISA kits (R&D). The cytokines measured include those considered to represent the relevant inflammatory and regulatory factors of the Th-1/Th-2 balance in the allergic/asthmatic process, such as interferon gamma (IFNγ), interleukins 4, 5, 10, 12, 13 (IL-4, IL-5, IL-10, IL-12, IL-13) and tumor necrosis factor alpha (TNFα).

A phase I/IIa double-blind controlled pilot study is performed in about 60 patients in the US and 20 in Israel, with moderate/severe, non steroid-dependent, asthma patients. The PA composition is administered orally in a single dose during one month, without changing the current anti-asthmatic medications of the patients. Follow-up continues for another month without treatment. A total of three on-site visits are planned on days 0, 30, 60. Blood samples are drawn in each visit to validate repetition of the safety profile of the PA composition. At the end-points an asthma symptom score, the need for concomitant medications and pulmonary functions, such as PEF or FEV1 are determined.

1. A pharmaceutical composition for the treatment or prevention of a disease or disorder in a tissue associated with an increase of eosinophils in said tissue, said composition comprising an effective amount of phosphatidic acid (PA) and a pharmaceutically acceptable carrier, diluent or excipient.

2. The composition according to claim 1, wherein said disease or disorder is eosinophilia or a related state.

3. The composition according to claim 1, wherein said disease or disorder is of the respiratory tract.

4. The composition according to claim 3, wherein said disease or disorder of the respiratory tract is asthma or asthma related state.

5. The composition according to claim 1, wherein said tissue is tissue of the respiratory tract.

6. The composition according to claim 5, wherein said tissue is blood.

7. The composition according to claim 4, wherein said asthma related state is chronic bronchial inflammation.

8. The composition according to claim 1, wherein said composition comprises at least about 5% PA.

9. The composition according to claim 8, wherein said composition comprises between about 5% to about 75%.

10. The composition according to claim 9, wherein said composition comprises between about 7% to about 50% PA.

11. The composition according to claim 10, wherein said composition comprises 10% to about 50% PA.

12. The composition according to claim 11, wherein said composition comprises between about 15% to about 30% PA.

13. The composition according to claim 12, wherein said composition comprises between about 15% to about 25% PA.

14. The composition according to claim 1, further comprising at least one other phospholipids.

15. Use of PA for the preparation of a pharmaceutical composition for the treatment or prevention of a disease or disorder in a tissue associated with an increase of eosinophils in said tissue.

16. The use according to claim 15, wherein said disease or disorder is eosinophilia or a related state.

17. The use according to claim 15, wherein said disease or disorder is of the respiratory tract.

18. The use according to claim 17, wherein said disease or disorder of the respiratory tract is asthma or asthma related state.

19. The use according to claim 15, wherein said tissue is tissue of the respiratory tract.

20. The use according to claim 19, wherein said tissue is blood.

21. The use according to claim 18, wherein said asthma related state is chronic bronchial inflammation.

22. The use according to claim 15, wherein said composition comprises at least about 5% PA.

23. The use according to claim 22, wherein said composition comprises between about 5% to about 75%.

24. The use according to claim 23, wherein said composition comprises between about 7% to about 50% PA.

25. The use according to claim 24, wherein said composition comprises 10% to about 50% PA.

26. The use according to claim 25, wherein said composition comprises between about 15% to about 30% PA.

27. The use according to claim 26, wherein said composition comprises between about 15% to about 25% PA.

28. The use according to claim 15, further comprising at least one other phospholipids.

29. A method for the treatment or prevention of a disease or disorder in a subject's tissue associated with an increase of eosinophils in said tissue, said method comprising administering to said subject in need thereof an effective amount of a composition comprising phosphatidic acid (PA).

30. The method according to claim 29, wherein said disease or disorder is eosinophilia or a related state.

31. The method according to claim 29, wherein said disease or disorder is of the respiratory tract.

32. The method according to claim 31, wherein said disease or disorder of the respiratory tract is asthma or asthma related state.

33. The method according to claim 29, wherein said tissue is tissue of the respiratory tract.

34. The method according to claim 33, wherein said tissue is blood.

35. The method according to claim 34, wherein said asthma related state is chronic bronchial inflammation.

36. The method according to claim 29, wherein said composition comprises at least about 5% PA.

37. The method according to claim 36, wherein said composition comprises between about 5% to about 75%.

38. The method according to claim 37, wherein said composition comprises between about 7% to about 50% PA.

39. The method according to claim 38, wherein said composition comprises 10% to about 50% PA.

40. The method according to claim 39, wherein said composition comprises between about 15% to about 30% PA.

41. The method according to claim 40, wherein said composition comprises between about 15% to about 25% PA.

42. The method according to claim 29, further comprising at least one other phospholipids.

43. A method for the prevention of an asthmatic state in a mammalian subject, said method comprising administering to a subject in need thereof an effective amount of a composition comprising phosphatidic acid (PA).

44. A method for reducing eosinophil concentration in a tissue of a subject, said method comprising administering to said subject with an effective amount of a composition comprising PA.

45. The method according to claim 44, wherein said subject suffers from eosinophilia or a related state.

46. The method according to claim 44, wherein said subject suffers from asthma or a related state.

47. The method according to claim 44, wherein said subject is human.

48. A method for preventing an increase in eosinophils in a tissue of a subject, said method comprising administering to said subject an efficient amount of a composition comprising PA.

49. The method according to claim 48, wherein said subject has an environmental or genetic predisposition to develop eosinophilia or a related state.

50. The method according to claim 48, wherein said subject has an environmental or genetic predisposition to develop asthma or a related state.

51. A method for reducing the accumulation of eosinophils in a tissue of the respiratory tract of a subject suffering from eosinophilia or a related state or having an environmental or genetic predisposition to develop eosinophilia, said method comprising administering to said subject an effective amount of a composition comprising PA.

52. The method according to claim 51, wherein said tissue is the lungs.

53. The method according to claim 51, wherein said tissue is the mucosa of the respiratory tract.

54. The method according to claim 51, wherein said eosinophilia or a related state is asthma.

55. The method according to claim 29, wherein said composition is administered orally.
